(19) United States
(12) Patent Application Publication
(10) Pub. No.: US 2006/0241602 A1
(43) Pub. Date: Oct. 26, 2006

(54) Hooked transverse connector for spinal implant system

(76) Inventor: Roger P. Jackson, Prairie Village, KS (US)

Correspondence Address: John C. McMahon, PO Box 30069, Kansas City, MO, 64112, US

(21) Appl. No.: 11/452,503

(22) Filed: Jun. 14, 2006

Related U.S. Application Data
(63) Continuation of application No. 11/187,417, filed on Jul. 22, 2005.
(63) Continuation-in-part of application No. 11/110,405, filed on Apr. 20, 2005.
(63) Continuation-in-part of application No. 09/588,924, filed on Jun. 6, 2000, now Pat. No. 6,884,244.

(51) Int. Cl. A61F 2/30

(52) U.S. Cl. 606/61

(57) ABSTRACT

A transverse connector assembly for connecting a pair of longitudinal connecting members of a spinal implant system includes a bendable arm with a hook member on either end thereof. Each hook member includes a bore and a curved receiving surface or pair of surfaces. The assembly includes a set screw with a break-off portion with a driving feature, and an axis of rotation corresponding to the bore central axis when the screw is rotatingly received in the bore. The set screw body also has a removal head and a radially extending structure on the break-off portion prohibiting engagement of the removal head by a driving tool while engaged with the break-off portion. The receiving surface of each hook member is oriented with respect to the set screw bore central axis for on axis engagement with a longitudinal connecting member. The hook members may be oriented toward one another, in the same direction or opposite one another. The set screw includes a projection that may be a point, a rim or a knurled dome.

### CROSS REFERENCE TO RELATED APPLICATION

This is a continuation-in-part of U.S. patent application Ser. No. 11/187,417, filed Jul. 22, 2005 and a continuation-in-part of U.S. patent application Ser. No. 11/110,405, filed Apr. 20, 2005, that is a continuation-in-part of U.S. patent application Ser. No. 09/588,924, filed Jun. 6, 2000, now U.S. Pat. No. 6,884,244, all of which are incorporated by reference herein.

### BACKGROUND OF THE INVENTION

The present invention is directed to transverse connectors for use between longitudinal connecting members, such as elongate rods, for placement along a patient's spine and to set screws for use in conjunction with such transverse connectors.

Surgically implanted spinal systems often include a pair of longitudinal connecting members that are typically in the form of elongate cylindrical rods secured along at least a portion of the spine and on opposite sides thereof by a system of bone attachment structures such as bone screws, hooks, or other bone anchors. Such an implant system provides support to a patient's spine to compensate for disease, injury or congenital defects. Transverse connectors typically attach to both of the longitudinal connecting members for stabilizing and adjusting alignment in such a system.

A preferred arrangement in such a system would be for the longitudinal connecting members to be in parallel alignment, but such is seldom the case. Placement of the longitudinal connecting members in a skewed alignment is often necessary to produce a desired corrective or supporting force on the spine. In practice, the pair of longitudinal connecting members may be co-planar, but not parallel. Also, for example, the longitudinal connecting members may be non-planar and transversely divergent. Each of these non-parallel configurations results in challenges and potential difficulties with respect to joining the transverse connector with the longitudinal connecting members, since a desired stable connection between the connector and the longitudinal connecting members most often occurs when ends of such a connector are securely attached to a longitudinal connecting member by a set screw or other attachment mechanism. If the longitudinal connecting members themselves are not parallel, the transverse connector may not form a desired stable juncture with the longitudinal connecting member, particularly if the transverse connector is rigid or adjustable in only one direction. In addition, the location of the spinal system sometimes requires that a transverse connector have an arch that is adjustable for clearing the spine or portions of the system. However a competing desire is that the connector does not protrude further rearward of the spine than is necessary.

Thus it is desirable for a transverse connecter to readily permit variations in the alignment of the longitudinal connecting members while providing relatively easy, fast and secure locking engagement to a rod or other longitudinal connecting member that is resistant to slipping or dislocation. Consequently, it is desirable to be able to frictionally engage the transverse connector with a rod or other longitudinal connecting member with a set screw or other closure system that utilizes high torquing forces during installation, results in a relatively low profile connector subsequent to installation, and has sufficient structure such that removal of the set screw or other closure mechanism is possible should such removal be desirable or necessary at a later time.

### SUMMARY OF THE INVENTION

A transverse connector assembly for a spinal implant system includes an arm with at least one hook member, the hook member having a set screw receiving aperture and a longitudinal connecting member receiving surface. The transverse connector is oriented such that the receiving surface of the hook member cooperates with the respective hook member aperture to position a longitudinal connecting member, such as a rod, so that a central axis of the longitudinal connecting member substantially intersects with the axis of rotation of a cooperating set screw when the longitudinal connecting member is fixed against the receiving surface by the set screw. The receiving surface is typically curvate and may also be in the form of a pair of spaced surfaces or strips.

The assembly further includes a set screw having a body, and may include a break-off portion with a driving feature. The set screw has a central axis of rotation when the body is rotatingly received in the hook member aperture. The set screw body also includes a removal head. The set screw that is equipped with a break-off portion further includes a radially extending structure on the break-off portion prohibiting engagement of the removal head by a driving tool while engaged with the break-off portion. Thus, a driving tool cannot be accidently used to drive both heads and over-torque the set screw upon installation. The projection, that may be in the form of a rim, is removed with the driving head, allowing for the same driving tool to be used to mate with the removal head for rotating the set screw out of the hook member.

Devices according to the invention typically include a pair of hook members disposed on either side of the arm. In certain embodiments according to the invention, the hook members are integral with the arm and the arm is bendable. In various embodiments according to the invention, the hook members open toward one another, away from one another, or in the same direction. Furthermore, in certain embodiments each set screw receiving aperture, that is in the form of a bore with a central axis, is oriented such that each bore central axis is perpendicular to a central axis of the arm in an unbent orientation, or at least to a portion of the arm that is fixed to the hook member when the arm is in a bent orientation. In other embodiments, each bore central axis intersects the arm axis at an oblique angle. Furthermore, in an embodiment that is useful when the arm is short such that the set screw break-off heads might interfere with one another, the hook member bores are disposed in skewed relationship, allowing for some clearance between set screws when the arm is bent or unbent.

Set screws according to the invention further include at least one projection for engagement with a longitudinal connecting member. The projection may be in the form of a point, a point and lateral rim combination, or a dome-shaped projection that may be knurled.

### OBJECTS AND ADVANTAGES OF THE INVENTION

Therefore, objects of the present invention include: providing a transverse connection system for spinal longitudinal connecting members that readily accommodates variations in degree of divergence and skew between the longitudinal connecting members; providing such a system to form a connection between spinal longitudinal connecting members such as rods positioned where a direct or straight connection between the rods is possible and also when such connection is obstructed by bone or other members of a spinal system; providing such a connection system between non-planar and/or non-parallel longitudinal connecting members; providing a closure or set screw for such a system that locks a transverse connector with respect to a longitudinal connecting member against both rotational and axial movement and provides for removal of the closure or set screw should removal be necessary; providing such a set screw having a plug body that is sized and shaped to be mateably received in threads of the transverse connector; providing such a set screw that includes an end for engaging a rod, the end having at least one of a point, a rim, and a dome and wherein the dome may be knurled; providing such a set screw that includes a driving head that breaks away at a predetermined torque to provide a comparatively low profile connector; providing such a set screw that includes a removal head that remains with the set screw subsequent to breakaway of the driving head; providing structure such that a socket tool utilized for torquing the driving head cannot be inadvertently engaged with the removal head to over torque the set screw upon installation; and providing such a set screw and an overall transverse connection system that is relatively easy to use, inexpensive to produce and especially well adapted for the intended usage thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a perspective view of a transverse connection assembly according to the present invention including a transverse connector and a pair of set screws, shown with portions of two cooperating longitudinal connecting members in phantom.

FIG. 2 is a fragmentary posterior view of a spinal column with the assembly of FIG. 1 shown in top plan and engaging a pair of cooperating longitudinal connecting members illustrated as solid rods.

FIG. 3 is an enlarged front elevational view of the assembly of FIG. 1.

FIG. 4 is a partial cross-sectional view taken along the line 4-4 of FIG. 2, showing a single hook of the transverse connector and a cooperating rod in cross section and further showing the cooperating set screw in front elevation and spaced from the connector and with an installation tool engaging a break-off head of the screw, the installation tool illustrated with portions broken away to show detail thereof.

FIG. 5 is a partial cross-sectional and front elevational view similar to FIG. 4, showing the set screw engaging the rod and also removal of the break-off head of the set screw.

FIG. 6 is a partial cross-sectional and front elevational view similar to FIGS. 4 and 5, showing removal of the set screw with the installation tool.

FIG. 7 is an enlarged front elevational view of a second embodiment of a transverse connection assembly according to the present invention including a transverse connector and a pair of set screws, shown in a first position for initial contact with portions of two cooperating longitudinal connecting members and the set screws being unengaged.

FIG. 8 is an enlarged front elevational view of the assembly of FIG. 7 showing the connector bent into a second position for engagement with the two cooperating longitudinal connecting members and further shown with the set screws fully engaged with the connecting members.

FIG. 9 is a fragmentary posterior view of a spinal column with two assemblies according to FIG. 7 shown in top plan view with the pair of cooperating longitudinal connecting members, also in top plan view.

FIG. 10 is an enlarged top plan view of a third embodiment of a transverse connector according to the invention.

FIG. 11 is an enlarged front elevational view of the connector of FIG. 10.

FIG. 12 is a cross-sectional view taken along the line 12-12 of FIG. 10.

FIG. 13 is an enlarged front elevational partially exploded view of the connector of FIG. 10, similar to FIG. 11, but with the connector being bent and also being shown with a pair of set screws.

FIG. 14 is a cross-sectional view taken along the line 14-14 of FIG. 13.

FIG. 15 is an enlarged front elevational view of the connector of FIG. 10, similar to FIG. 13, being shown implanted on spaced, non-coplanar longitudinal connecting members.

FIG. 16 is an enlarged front elevational view, similar to FIG. 15, with portions broken away to show detail thereof, showing the set screws engaged with the non-coplanar longitudinal connecting members.

FIG. 17 is a cross-sectional view taken along the line 17-17 of FIG. 16.

FIG. 18 is an enlarged top plan view of a fourth embodiment of a transverse connector according to the invention.

FIG. 19 is an enlarged front elevational view of the connector of FIG. 18.

FIG. 20 is a cross-sectional view taken along the line 20-20 of FIG. 18.

FIG. 21 is an enlarged top plan view of a fifth embodiment of a transverse connector according to the invention.

FIG. 22 is an enlarged front elevational view of the connector of FIG. 21.

FIG. 23 is a cross-sectional view taken along the line 23-23 of FIG. 21.

### DETAILED DESCRIPTION OF THE ILLUSTRATED EMBODIMENTS

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is noted that any reference to the words top, bottom, up and down, and the like, in this application refers to the alignment shown in the various drawings, as well as the normal connotations applied to devices according to the invention, and is not intended to restrict the positioning of bone screws, longitudinal connecting members and transverse connectors in actual use.

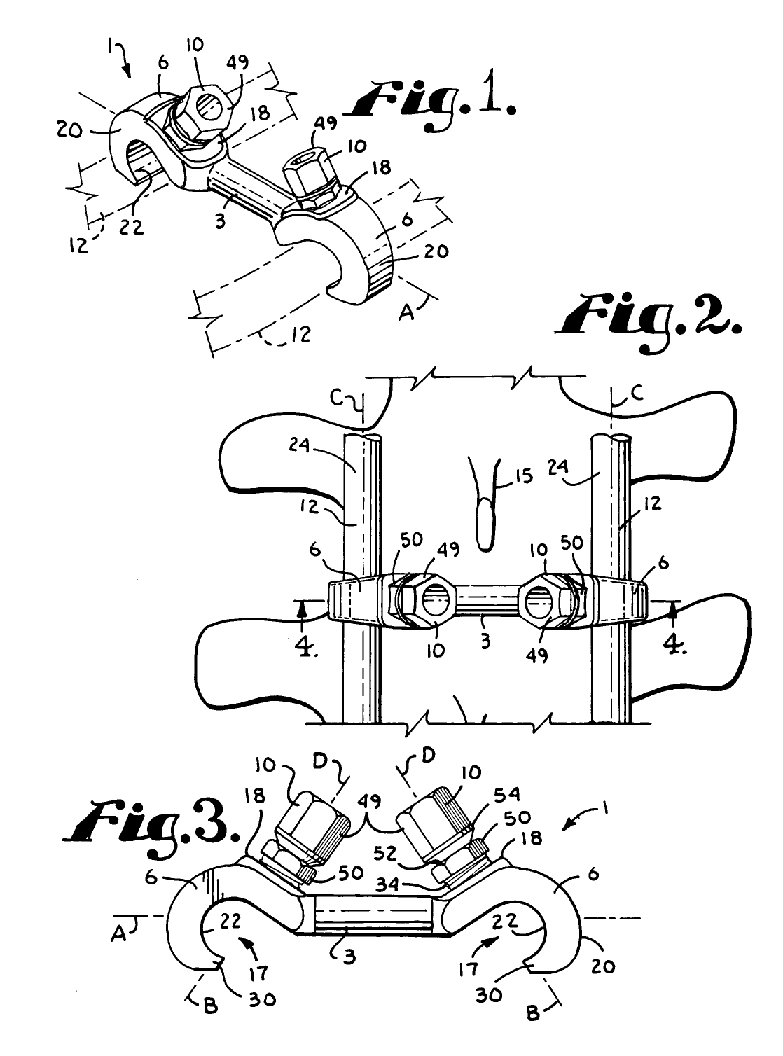

With reference to FIGS. 1-6, the reference numeral 1 generally designates a transverse connector device or assembly having an arm 3, a pair of hook members 6 and a pair of closure members or set screws 10. The assembly 1 typically is mounted on a pair of longitudinal connecting members such as rods 12 that are implanted on a spine 15 utilizing bone fixation structures that may include monoaxial or polyaxial bone screws, hooks, or other fixation devices and combinations thereof.

The hook members 6 are disposed at either end of the arm 3. In the embodiment illustrated in FIGS. 1-6, the hook members 6 are integral with or otherwise fixed to the arm 3 and are positioned substantially opposite one another along a central longitudinal axis A of the arm 3 with rod-receiving openings 17 of the hook members 6 facing one another. Each hook member 6 includes a set screw receiver portion 18 and a c-shaped portion 20 having a rod bearing surface 22 shaped to substantially cradle a portion of an outer surface 24 of a rod 12 with a first radius of the surface 22 being slightly larger than a second radius of the outer surface 24 of the rod 12.

Each receiver portion 18 has an opening or through bore 26 formed therein with a helical thread 27 or other helically wound guide and advancement structure sized and shaped to rotatingly receive and mate with one of the set screws 10. Each bore 26 has a central axis B corresponding to an axis of rotation of a cooperating set screw 10. In the embodiment shown in FIGS. 1-6, the assembly 1 is substantially symmetrical with both of the bore 26 central axes B and the axis A being in substantially the same plane. Each hook member c-shaped portion 20 includes a lip 30 that extends beyond a respective axis B in a direction towards the arm 3 and thus is shaped to extend about a cooperating cylindrical rod 12 a distance beyond a substantially central longitudinal axis C thereof when the rod 12 is cradled by the c-shaped portion 20 and positioned adjacent the surface 22. When the set screw 10 engages the rod 12, fixing the assembly 1 to the rod 12, the axis B and the axis C intersect or nearly intersect, providing for a substantially on-axis engagement of a set screw 10 and the rod 12, with the set screw 10 having a central axis of rotation D intersecting or nearly intersecting the axis C of the rod 12. It is noted that because the rod 12 will often be curved, the axis C will also be curvate, resulting in a somewhat approximate on-axis relationship between the set screw and the rod. In the embodiment illustrated in FIGS. 1-6, the axis B of each bore 26 and the axis C of the cooperating rod 12 are substantially perpendicular to one another while the axis B forms an oblique angle with the arm axis A.

It is also noted that typically a surgeon will have available a plurality of transverse connector assemblies 1 of different lengths, as measured along the arm axis A, to accommodate different sized patients and spinal apparatus being placed at different locations along the length of the spine, resulting in various distances between the rods 12. For those instances when the desired distance between two rods 12 is between the sizes of the devices 1 available to the surgeon, the device 1 may be bent at the arm 3 to provide a slightly shorter transverse connector.

With reference to FIGS. 1, 2 and 4-6, the longitudinal connecting members or rods 12 for use with devices according to the invention are typically elongate, often curved, rod or elongate rod-like members that generally extend between multiple bone screws or other elements of a spinal system. The illustrated rods 12 are circular in cross section and have a smooth external surface; however it is foreseen that longitudinal connecting members having other types of cross section and having rough or knurled external surfaces could be utilized.

As illustrated in FIGS. 4-6, the closures or set screws 10 threadably engage the set screw receiver portions 18 and frictionally engage a respective rod 12 disposed adjacent the rod bearing surfaces 22 in an on-axis orientation, thereby clamping the rod 12 to the assembly 1. Each of the closures or set screws 10 includes a body 34 that has a generally cylindrical or plug shape with a substantially circular horizontal cross section and a central axis of rotation D that is the same as the axis B when the set screw 10 is rotatingly mated in the bore 26. Located at a substantially flat or planar lead or bottom end surface 36 of the set screw 10 opposite a substantially flat top surface 37 is a point or small domed region 38 that projects outwardly from the surface 36 along the axis D (downwardly in FIGS. 4-6). Also projecting out from the surface 36 in the same direction as the point 38 is a rim 40 that encircles the point 38 and is disposed near the threaded periphery of the body 34. A dome shaped projection, as illustrated in FIGS. 8 and 13 and will be discussed in greater detail below, may replace the point and rim projections of the device 1. It is foreseen that such a dome in accordance with the invention may cover most or a portion of the bottom surface 36 and may vary in radius of generation or curvature. It is also foreseen that in certain embodiments, a domed bottom may have an apical point or may be at least partially covered with knurling or the like to provide additional gripping during usage.

Located on the cylindrical set screw body 34 is a guide and advancement structure or thread 44 mateable with the thread 27 of the set screw receiver portion 18. In the illustrated embodiment, the guide and advancement structure 44 is a V-thread. The thread 44 acts cooperatively with the set screw receiver portion 18 to allow the screw 10 to be inserted into and rotated relative to the receiver portion 18 and to guide and advance the set screw 10 along the axis B as the screw 10 is rotated clockwise or in a reverse direction when rotated counterclockwise. The guide and advancement structure 44 and cooperating structure 27 may also be splay resistant, such as a helically wound reverse angle thread form described in U.S. patent application Ser. No. 11/110,405, filed Apr. 20, 2005, and incorporated by reference herein. It is also foreseen that other types of guide and advancement structure could be utilized, such as, for example, a buttress thread form, a square thread form or some other type of structure such as a flange form.

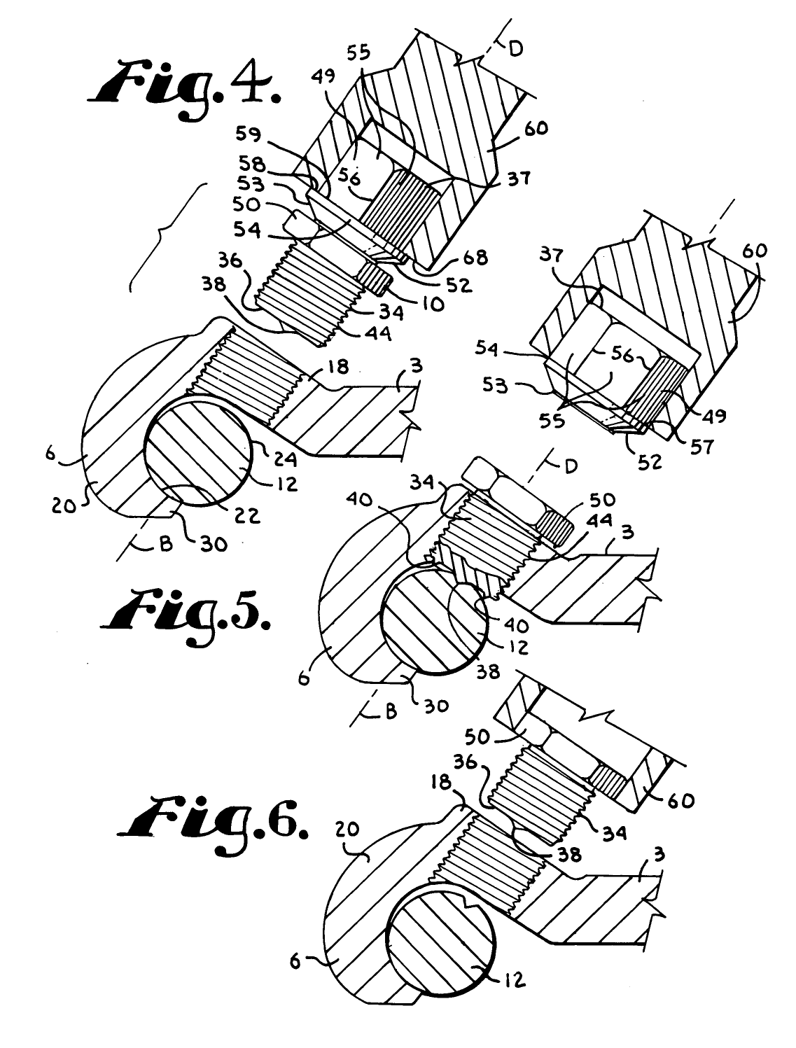

Each closure or set screw 10 also includes a driving or installation break-off head 49 and a removal head 50 that are coaxially attached to the body 34. The removal head 50 is located between the body 34 and the driving head 49 and disposed near the thread 44. The driving head 49 is disposed on a break-off portion 52 that is secured to the body 34 at a breakaway region 53 adjacent the removal head 50 and is designed to break away from the remainder of the set screw 10 subsequent to a predetermined torque being applied to the driving head 49, such as 100 inch pounds, during installation of the set screw 10 into one of the receiver portions 18. A barrier in the form of a cylindrical rim stop 54 is disposed on the break-off portion 52 between the breakaway region 53 and the driving head 49. The illustrated rim stop 54 is adjacent to the driving head 49. As illustrated in FIGS. 3-5, the driving head 49 top surface 37, that is perpendicular to the axis of rotation D, is hexagonal and is formed by six flat faces 55 that are joined together in a hexagonal pattern. Each of the faces 55 runs parallel to the axis of rotation D. Together, the faces 55 form a hexagonal driving feature adapted for use with a socket type driving tool. The illustrated rim stop 54 is substantially circular in cross-section and concentric with the driving head 49. As shown in FIG. 4, edges 56 formed by the six flat faces 55 of the driving head 49 define in part an outer edge or circumference 57 of the rim stop 54, with the rim stop 54 forming a discontinuous annular surface 58 between each of the edges 56 at a lower end or base 59 of the driving head 49.

An installation and removal tool 60 is provided for driving and torquing the driving head 49 and if needed or desired, driving the removal head 50. The installation/removal tool 60 is a socket-type tool and is described, for example, Applicant's U.S. patent application Ser. No. 11/110,405, filed Apr. 20, 2005, incorporated by reference herein. In use, the discontinuous annular surface 58 of the rim stop 54 abuts against a bottom surface 68 of the tool 60 when the tool engages the driving head 49, providing a barrier so that the installation/removal tool 60 cannot inadvertently grip the removal head 50 when installing the set screw 10 and thereby produce too much torque by bypassing the torque limitation associated with the break-off driving head 49.

FIG. 5 shows the driving head 49 and the rim stop 54 broken away from the body 34. Although the illustrated rim stop 54 is substantially cylindrical and therefore the circumference 57 is circular, it is foreseen that the rim stop may be of a variety of other geometries that extend outwardly from one or more faces 55 of the driving head 49 so that the tool 60 abuts the stop and does not engage with the removal head 50 when engaged with the driving head 49.

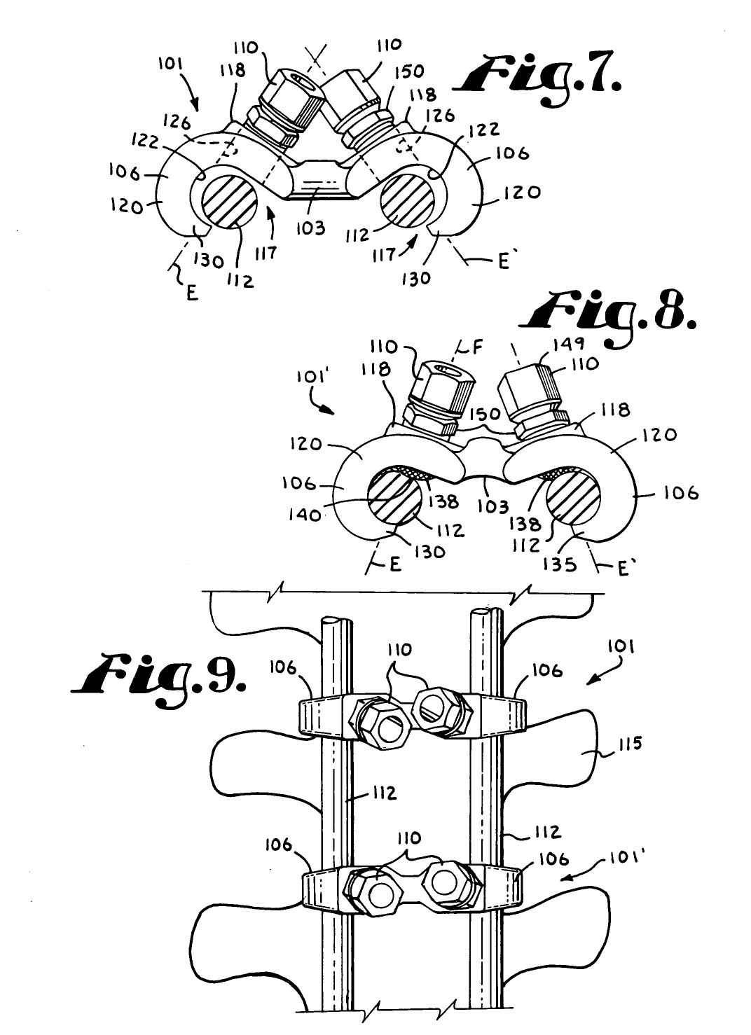

The illustrated removal head 50 has a hexagonal cross-section substantially identical in size and shape to the driving head 49 so that the same tool 60 may be used for both installation and removal. In the illustrated embodiment the removal head 50 is integral with the set screw body 34. If, subsequent to installation, it is necessary or desirable to remove the set screw 10, the tool 60 may be used as illustrated in FIG. 6. The socket of the tool 60 is snugly mateable with the removal head 50 to allow the set screw 10 to be rotated and removed from the set screw receiver portion 18. Because removal usually takes less torque in comparison to installation, the removal head structure does not need to be as tall as the head 49. It is also foreseen that if desired, the head 50 could be smaller or larger than the head 49, requiring a different removal tool (not shown) with a smaller or larger socket.

It is foreseen that a set screw according to the invention may include a removal head and driving head combination similar to what is shown in FIGS. 1-6, but with the driving feature of the driving head being an imprint or other concave, internal driving formation formed in a top surface of the driving head as illustrated in Applicant's U.S. patent application Ser. No. 11/187,417, filed Jul. 22, 2005, the disclosure of which is incorporated by reference herein. In such an embodiment, the removal head would be disposed on or adjacent a body or plug portion of the closure, and the driving feature would be spaced from the removal head and disposed on a break-off portion of the closure. Thus, the removal head having a radially outwardly extending driving surface would be positioned axially between the closure body and the internal driving feature. However, the removal head could not be accessed until the break-off portion is first removed, utilizing the external or internal driving feature for the removable portion of the closure. It is also foreseen that in such an embodiment, the imprint or other internal driving formation in the driving head may be sized such that the same driving tool could be utilized to both rotate the internal driving head feature in the break-off portion, and subsequently the external removal head feature, after the break-off portion is removed.

In use, bone screws or other bone attachment members are implanted into vertebrae of the spine 15. Eventually rods 12 are attached to the bone screws and at least one and up to a plurality of connector assemblies 1 are linked to each of the rods at the c-shaped portions 20 by manipulating the rods 12 and the connector assembly 1 to result in a loosely hooked or connected configuration shown in FIGS. 1 and 2. Although FIG. 4 illustrates the set screw 10 being spaced from the set screw receiver portion 18, the set screws 10 are preferably pre-inserted into the receiver portions 18 as shown in FIG. 3 such that the leading surface 36 and point 38 and rim 40 are disposed within the bore 26 and do not extend into the opening 17. With reference to FIGS. 4-6, the installation/removal tool 60 is utilized to install the set screw 10 by engaging and rotating the driving head 49 during which installation the driving head 49 breaks from the body 34 of the set screw 10 at a predetermined torque so as to tightly snug the set screw 10 against the rod 12 with the point 36 and portions of the rim 40 engaging and penetrating the rod 12 at the surface 24. As is illustrated in FIG. 5, the set screw 10 axis D is substantially perpendicular to the longitudinal axis C of the rod 12, with the rod being securely held against the surface 22 of the c-shaped portion 20 of the hook member 6.

FIG. 5 illustrates the fully installed set screw 10 in a receiver portion 18 and capturing a rod 12 in the c-shaped portion 20. With reference to FIG. 6, if removal of the set screw 10 is required, the installation/removal tool 60 is again utilized to rotate and remove the set screw body 34 by engaging the removal head 50 and rotating the body 34 out of the receiver portion 18.

With reference to FIGS. 7-9, the reference numeral 101 generally represents a second or alternative embodiment of a transverse connector device according to the invention. The device includes an arm 103, a pair of hook members 106 and a pair of closure members or set screws 110. The assembly 101 typically is mounted on a pair of longitudinal connecting members such as rods 112 that are implanted on vertebrae 15 of a spine utilizing bone fixation structures that may include monoaxial or polyaxial bone screws, hooks, or other fixation devices and combinations thereof.

The device 101 is substantially similar to the device 1 with the hook members 106 being disposed at either end of the arm 103. The hook members 106 each include an opening 117 for receiving the rod 112, a set screw receiving portion 118 and a c-shaped portion 120 with a rod bearing surface 122, substantially similar to the opening 17, receiving portion 18, c-shaped portion 20 and rod bearing surface 22 previously described herein with respect to the device 1. However, bores 126 running through each of the receiving portions 118 do not have central axes disposed in substantially the same plane as do the bores 26 of the device 1. Because the arm 103 of the device 101 is shorter than the arm 3 of the device 1, to provide clearance for the set screws 110, the bores 126 for receiving the set screws 110 are disposed to allow for a side by side or overlapping placement of the set screws along the arm 103 as illustrated in FIG. 7. Stated in another way, a central axis E of the bore 126 disposed in one of the hook members 106 is disposed in a skewed non-planar relationship with a central axis E′ of the bore 126 disposed in the other opposed hook member 106.

Similar to the device 1, the device 101 c-shaped portions 120 each include a lip 130 that extends beyond the axis E or E′ in a direction towards the arm 103. The set screws 110 are also substantially similar to the set screws 10, with one exception: the point 38 and the rim 40 are replaced by a knurled dome 138 having an apex 140 that extends along an axis F of the set screw 110.

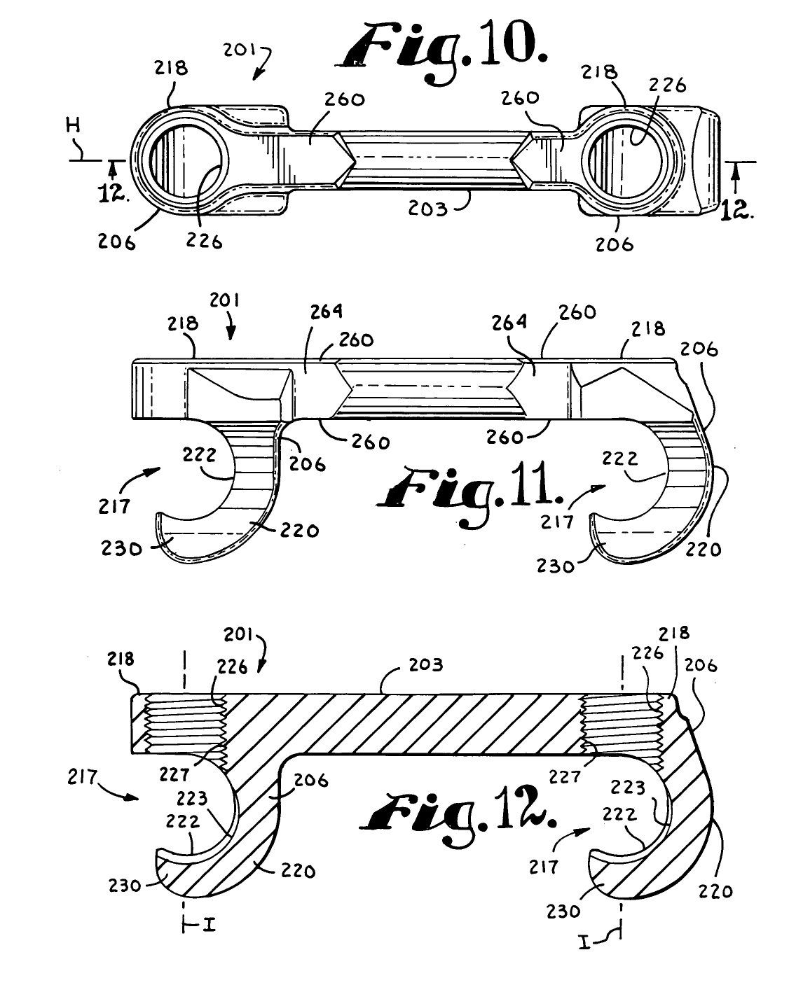

FIGS. 7-9 illustrate a situation in which the rods 112 are located in very close proximity to one another and the device 101 is initially slightly longer (length taken along the arm 103) than required for clamping of both of the rods 112 by the device 101 in a desired position. Therefore, the device 101 is initially positioned on the rods 112 as illustrated in FIG. 7 and then the arm 103 is bent slightly as shown in FIG. 8 to an orientation or position 101′, bringing the c-shaped portions 120 toward one another and the surfaces 122 snugly against each of the rods 112. FIG. 9 illustrates one device 101 prior to bending and another device 101′ that has been bent. Bending of the device provides a further advantage of moving the bone screw break-off heads away from one another, providing additional clearance when tightening the screws 110 against the rods 112.

The set screws 110 are advanced into the bores 126 similar to what has been previously described with respect to the set screws 10 of the device 1 until the break off heads 149 are removed, leaving removal heads 150 exposed for use in removing the set screw 110, if desired. As illustrated in FIG. 8, when each of the set screws 110 engage a respective rod 112, fixing the assembly 101 to the pair of rods 112, with the knurled domes 138 engaging cylindrical surfaces of the rods 112, each set screw 110 is in a substantially on axis relationship with the respective rod 112, similar to the rod and screw cooperation previously described herein with respect to the device 1.

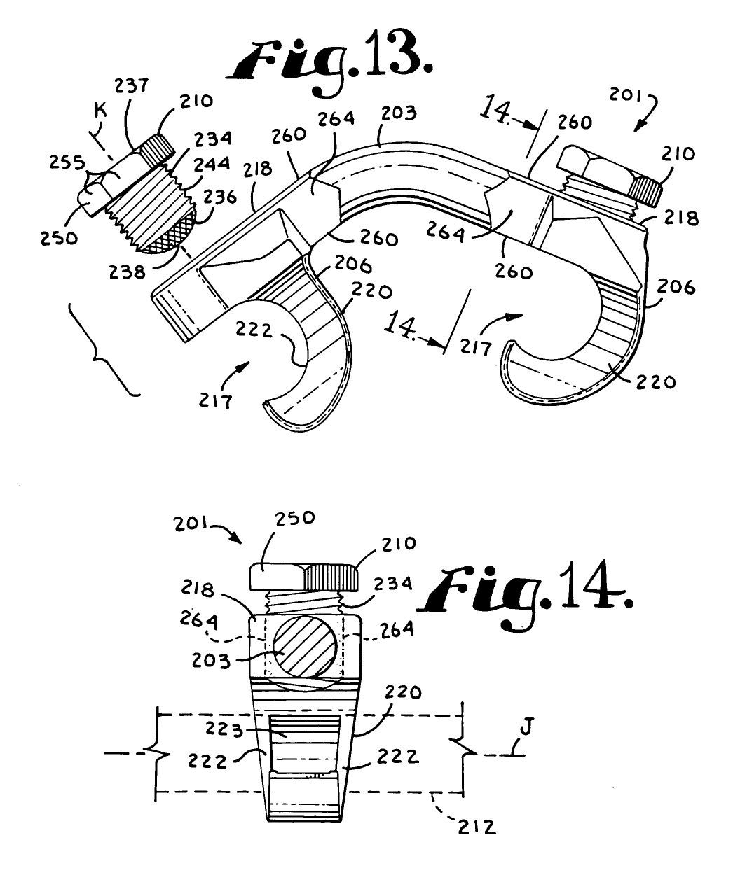

With reference to FIGS. 10-17, a third embodiment of a transverse connector device according to the invention, generally 201, is illustrated having an arm 203, a pair of hook members 206 and a pair of closure members or set screws 210. The device 201 is substantially similar to the devices 1 and 101 previously described herein. Furthermore, the device 201 is mountable on a pair of longitudinal connecting members, such as rods 212, by inserting the device 201 laterally to both of the rods 212 and moving the device 201 in a direction toward both of the rods 212 until both rods are engaged by hook members 206 of the device 201. This is made possible because the hook members 206 that are disposed at either end of the arm 203 have openings 217 directed in substantially the same direction. In the embodiment illustrated in FIGS. 10-17, the hook members 206 are integral with or otherwise fixed to the arm 203 at either end thereof and positioned along a central longitudinal axis H of the arm 203. Each hook member 206 includes a set screw receiver portion 218 and a c-shaped portion 220 having a pair of spaced rod bearing surfaces or strips 222 shaped to engage and substantially cradle a portion of an outer surface 224 of a rod 212 with a first radius of the surfaces 222 being slightly larger than a second radius of the outer surface 224 of the rod 212. The surfaces 222 are best illustrated in FIGS. 14 and 17. The surfaces 222 are disposed on either side of an arcuate groove or recess 223 formed in each c-shaped portion 220.

Each receiver portion 218 has an opening or through bore 226 formed therein with a helical thread 227 or other helically wound guide and advancement structure sized and shaped to rotatingly receive and mate with one of the set screws 210. Each bore 226 has a central axis I corresponding to an axis of rotation of a cooperating set screw 210. The bore 226 central axes I and the axis H of the arm are in substantially the same plane. Each hook member c-shaped portion 220 includes a lip 230 that extends beyond a respective axis I and thus is shaped to extend about a cooperating cylindrical rod 212 a distance beyond a substantially central longitudinal axis J thereof when the rod 212 is cradled by the c-shaped portion 220 and positioned engaging the surfaces 222. When the set screw 210 engages the rod 212, fixing the assembly 201 to the rod 212, the axis I and the axis J intersect or nearly intersect, providing for a substantially on-axis engagement of a set screw 210 and the rod 212, with the set screw 210 having a central axis of rotation K intersecting or nearly intersecting the axis J of the rod 212. It is noted that because the rod 212 will often be curved, the axis J will also be curvate, resulting in a somewhat approximate and yet substantial on-axis relationship between the set screw and the rod. In the embodiment illustrated in FIGS. 10-17, the axis I of each bore 226 and the axis J of the cooperating rod 212 are substantially perpendicular to one another, and the axis I of the bore is also substantially perpendicular to the arm axis H, or at least to an axis of a portion of the arm 203 when the arm is bended at or near a central region thereof as illustrated in FIGS. 13, 15 and 16.

As illustrated in FIGS. 13-16, the closures or set screws 210 threadably engage the set screw receiver portions 218 and frictionally engage a respective rod 212 disposed adjacent the rod bearing surfaces 222 in an on-axis orientation, thereby clamping the rod 212 to the assembly 201. Each of the closures or set screws 210 includes a body 234 that has a generally cylindrical or plug shape with a substantially circular horizontal cross section and a central axis of rotation K that is the same as the axis I when the set screw 210 is rotatingly mated in the bore 226. A lead or bottom end surface 236 of the set screw 201 opposite a substantially flat top surface 237 is of a knurled dome shape similar to the dome 138 previously described herein with respect to the transverse connector 101. The dome 236 includes an apex 238 that projects along the axis K of the set screw.

Located on the cylindrical set screw body 234 is a guide and advancement structure or thread 244 mateable with the thread 227 of the set screw receiver portion 218. In the illustrated embodiment, the guide and advancement structure 244 is a V-thread. The thread 244 acts cooperatively with the set screw receiver portion 218 to allow the screw 210 to be inserted into and rotated relative to the receiver portion 218 and to guide and advance the set screw 210 along the axis I as the screw 210 is rotated clockwise or in a reverse direction when rotated counterclockwise. The guide and advancement structure 244 and cooperating structure 227 may also be splay resistant, such as a helically wound reverse angle thread form described in U.S. patent application Ser. No. 11/110,405, filed Apr. 20, 2005, and incorporated by reference herein. It is also foreseen that other types of guide and advancement structure could be utilized, such as, for example, a buttress thread form, a square thread form or some other type of structure such as a flange form.

Each closure or set screw 210 also includes a driving installation and removal head 250 coaxially with and integral or otherwise attached to the body 234. The driving head top surface 237, that is perpendicular to the axis of rotation K, is hexagonal and is formed by six flat faces 255 that are joined together in a hexagonal pattern. Each of the faces 255 runs parallel to the axis of rotation K. Together, the faces 255 form a hexagonal driving feature adapted for use with a socket type driving tool (not shown) that is similar to the installation and removal tool 60 previously described herein. In use, the set screw 210 may be installed and removed from the receiver portion 218 with the same driving tool. Similar to what was previously described with respect to the device 1, the set screws 210 are preferably pre-installed prior to implantation of the device 201, with the domed bottom 236 disposed within the bore 226 as illustrated in FIGS. 13-15. The set screw 210 may then be tightened against a rod 212 as illustrated in FIG. 16.

In FIGS. 13-16, the device 201 is illustrated in use wherein a straight connection is not possible, for example, when the transverse connector must extend between non-coplanar rods, skewed rods, across or over a posterior element of a vertebra and the spinal canal, or as described previously herein wherein a distance between the rods is smaller than an available length of a device 201. In such situations, the arm 203 may be bent, twisted or oriented by a combination of bending and twisting with tools to result in a desired distance between the hook members 206 and a desired position of such hook members for securely clamping to the pair of rods 212. When possible, the arm 203 further includes opposed top and bottom flat or planar surfaces 260 at either end thereof and opposed flat or planar side surfaces 264 at either end thereof, disposed adjacent each hook member 218 to allow for a bending tool (not shown) to grasp the arm 203 at either end thereof and at the opposed flats 260 or the opposed flats 264 to readily bend or twist the arm 203 to a desired orientation.

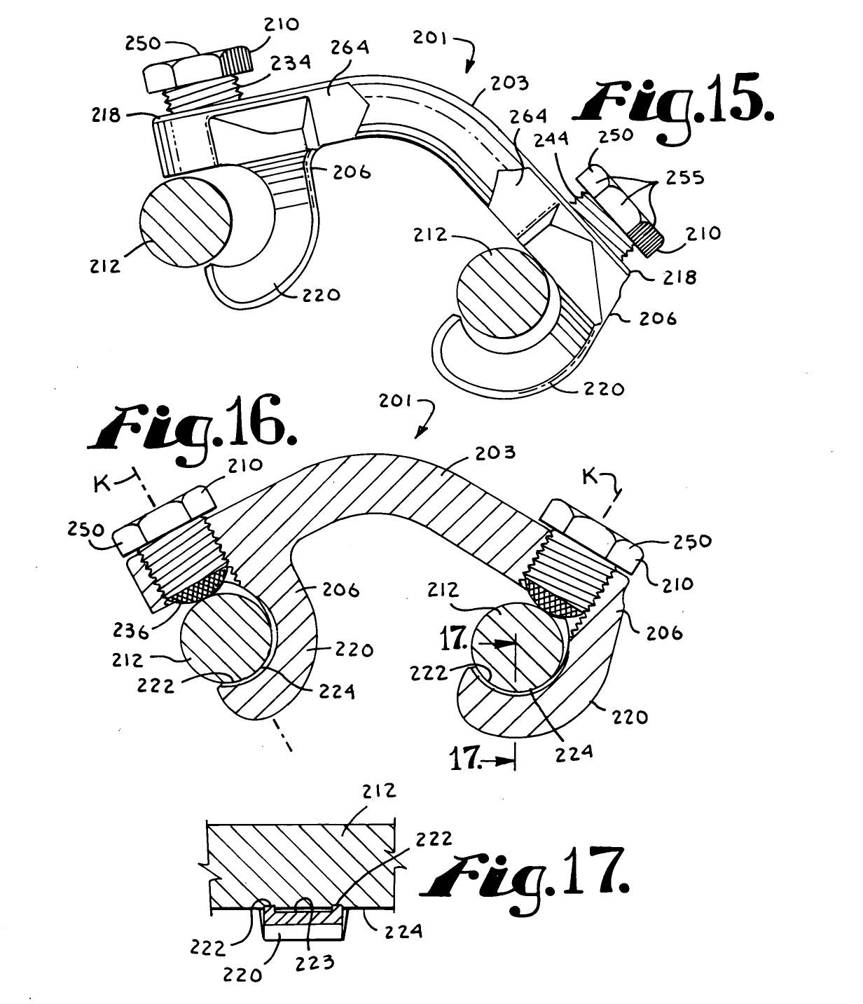

With particular reference to FIG. 15, the bent device 201 having hook members 206 opening in substantially the same direction, and equipped with pre-inserted set screws 210, may first be placed laterally of both the rods 212 and in close proximity thereto, allowing for a single lateral or slightly twisting movement of the device 201 to result in the simultaneous capture or hooking of both of the rods 212 into the openings 217 of the c-shaped portions 220, even if the rods are not in the same plane or are skewed with respect to one another. Then, a socket type installation/removal tool (not shown) is utilized to fully install the set screw 210 by engaging and rotating the driving head 250 until the dome 236 frictionally engages the rod surface 224, pressing the rod 212 against the pair of rod bearing surfaces 222. As is illustrated in FIG. 16, each set screw 210 axis K is substantially perpendicular to the longitudinal axis J of the respective rod 212, with the rod being securely held against the surfaces 222 of the c-shaped portion 220 of the hook member 206. If removal of the set screw 210 is required, the same socket-type tool used for installation may be used again to rotate and remove the set screw body 234 by engaging the head 250 and rotating the body 234 out of the receiver portion 218.

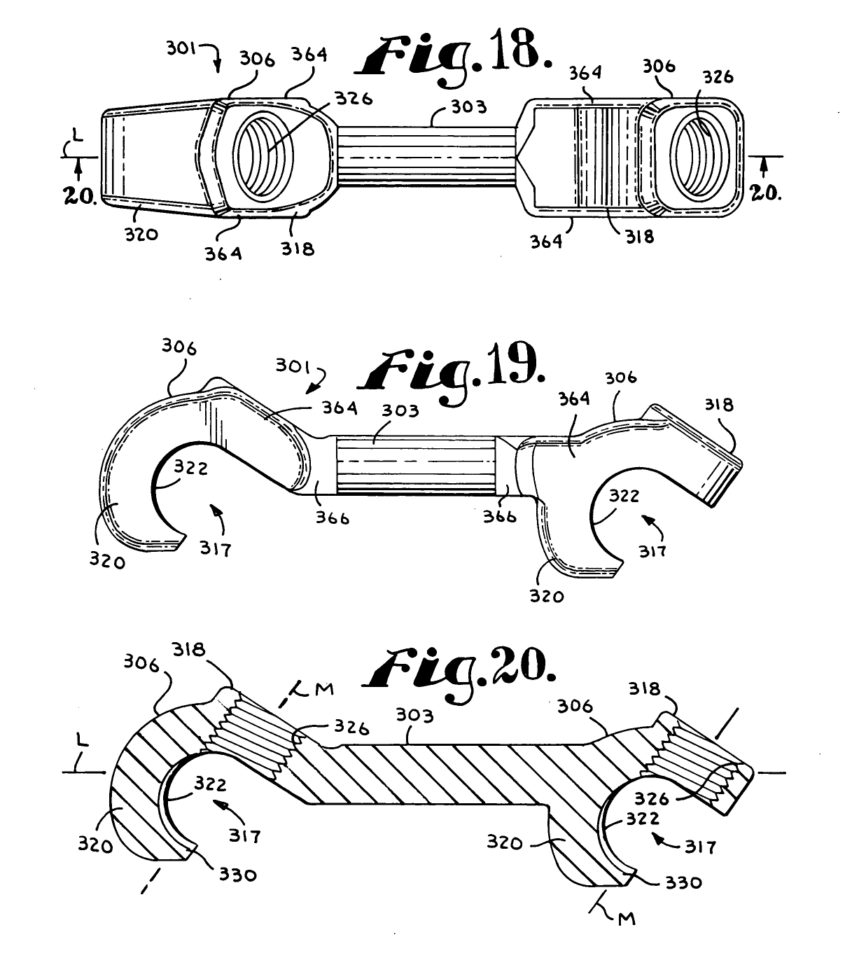

With reference to FIGS. 18-20, the reference numeral 301 generally represents a fourth alternative embodiment of a transverse connector device according to the invention. The device includes an arm 303 having a central axis L with a round, square or other cross-sectional shape, a pair of hook members 306 and a pair of closure members or set screws 10, 110 or 210 described previously herein. The device 301 is substantially similar to the device 201 with the hook members 306 being disposed at either end of the arm 303 and including substantially similarly directed openings 317 for receiving rods, such as the rods 212. Each hook member further includes a set screw receiving portion 318 and a c-shaped portion 320 with rod bearing surfaces 322 substantially similar to the receiving portion 218, c-shaped portion 220 and rod bearing surfaces 222 previously described herein with respect to the device 201. However, unlike the device 201, wherein the axis H is substantially perpendicular to the axis I, the device 301 includes threaded bores 326, each running through a respective receiving portion 318 and having a central axis M that is disposed at an oblique angle with respect to the arm axis L. The openings 317 are also advantageously directed somewhat downwardly, providing for ease in simultaneously capturing a pair of rods with a single lateral and slightly downward movement.

Similar to the devices 1, 101 and 201, the device 301 c-shaped portions 320 each include a lip 330 that extends beyond the axis M such that when a set screw bears down about a rod disposed against the surface 322, the rod engages and is cradled by the c-shaped portion 320. The c-shaped portions 320 of the device 301 further include opposed flat side surfaces 364 allowing for gripping of the device 301 to bend or twist the arm 303, if desired. Opposed flat side surfaces 366 and flat top and bottom surfaces may also be provided on the arm 303 near each of the hook members 306, to allow for holding during bending and/or twisting of the arm 303.

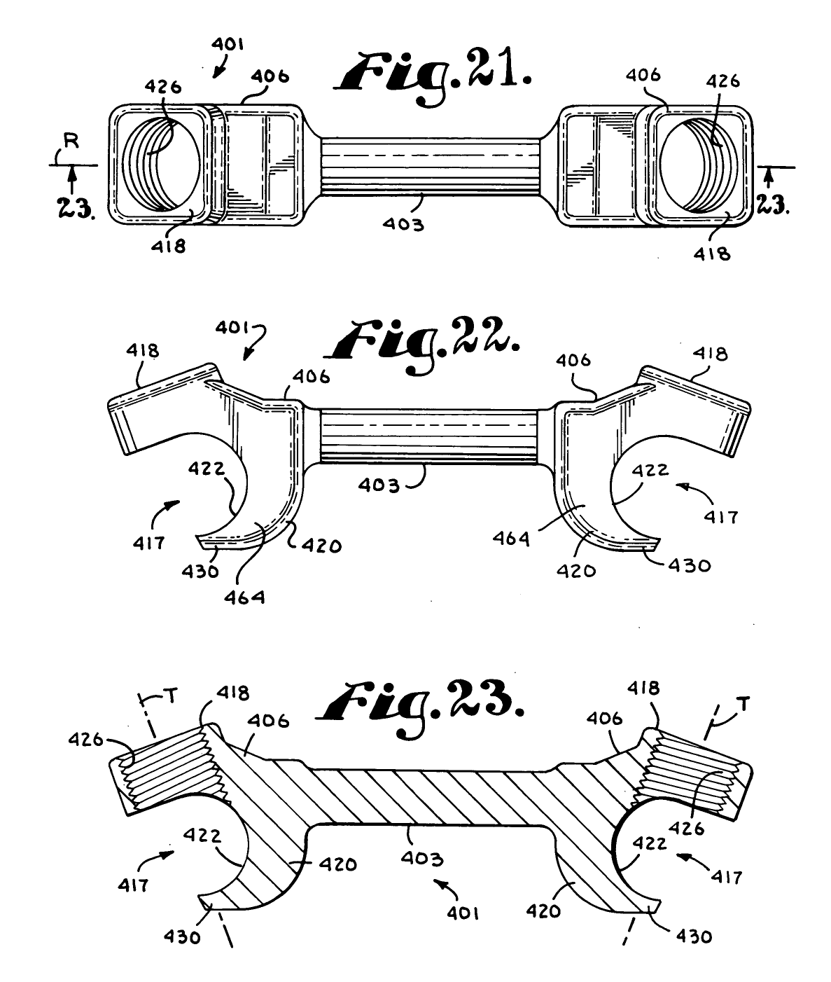

With reference to FIGS. 21-23, the reference numeral 401 generally represents a fifth alternative embodiment of a transverse connector device according to the invention. The device includes an arm 403 having a central axis R, a pair of hook members 406 and a pair of closure members or set screws 10, 110 or 210 described previously herein. The device 401 is substantially similar to the device 1 with the hook members 406 being disposed at either end of the arm 403. However, the device 401 is different from the device 1 with respect to openings 417 of the hook members 406. While the openings 17 of the members 6 face substantially toward one another, the hook member openings 417 are directed opposite one another, each opening 417 being directed outwardly in a direction substantially away from the arm 403. Similar to the device 1, each hook member includes a set screw receiving portion 318 and a c-shaped portion 320 with a rod bearing surface 322 substantially similar to the receiving portion 18, c-shaped portion 20 and rod bearing surface 22 previously described herein with respect to the device 1. The device 401 also includes threaded bores 426 running through each of the receiving portions 418, each having a central axis T that is disposed at an oblique angle with respect to the arm axis R. The openings 417 are also advantageously directed somewhat downwardly, providing for ease in capturing a pair of rods with a downward movement, particularly if the arm 403 is bent prior to installation.

Similar to the devices 1, 101, 201 and 301, the device 401 c-shaped portions 420 each include a lip 430 that extends beyond the axis T such that when a set screw bears down about a rod disposed against the surface 422, the rod engages and is cradled by the c-shaped portion 420. The c-shaped portions 420 of the device 401 further include opposed flat side surfaces 464 allowing for gripping of the device 401 to bend or twist the arm 403 if desired. Opposed flat side surfaces and opposed flat top and bottom surfaces may also be provided on the arm 403 near each of the hook members 406, to allow for holding during bending and/or twisting of the arm 403.

It is to be understood that while certain forms of the present invention have been illustrated and described herein, it is not to be limited to the specific forms or arrangement of parts described and shown.

What is claimed and desired to be secured by Letters Patent is as follows:

1. A device for connecting a pair of longitudinal connecting members of a spinal implant system, the device comprising:
a) an arm with at least one hook member, the hook member having a set screw receiving aperture and a longitudinal connecting member receiving surface;
b) a set screw having a body, a break-off portion with a driving feature, and a central axis of rotation when the body is rotatingly received in the hook member aperture, the body also having a removal head, the set screw further having a radially extending structure on the break-off portion prohibiting engagement of the removal head by a driving tool while engaged with the break-off portion; and wherein
c) the receiving surface of the hook member is oriented with respect to the hook member aperture to position a longitudinal connecting member such that a central axis of the longitudinal connecting member substantially intersects with the axis of rotation of the set screw when the longitudinal connecting member is fixed against the receiving surface by the set screw.

2. The device of claim 1 wherein the receiving surface is curvate.

3. The device of claim 1 wherein the receiving surface is a pair of spaced strips.

4. The device of claim 1 wherein the hook member is a first hook member disposed near a first end of the arm and further comprising a second hook member disposed near a second opposite end of the arm, the first and second hook members opening towards one another.

5. The device of claim 1 wherein the hook member is a first hook member disposed near a first end of the arm and further comprising a second hook member disposed near a second opposite end of the arm, the first and second hook members each opening in the same direction.

6. The device of claim 1 wherein the hook member is a first hook member disposed near a first end of the arm and further comprising a second hook member disposed near a second opposite end of the arm, the first and second hook members opening in opposite directions.

7. The device of claim 1 wherein the arm is bendable, the hook member is a first hook member, the set screw receiving aperture is a bore having a first axis, and further comprising a second hook member with a bore having a second central axis, the first and second axes being substantially parallel when the arm is in an unbent orientation.

8. The device of claim 1 wherein the arm is bendable, the hook member is a first hook member, the set screw receiving aperture is a bore having a first axis, and further comprising a second hook member with a bore having a second central axis, the first and second axes being in a skewed relationship when the arm is unbent and when the arm is bent.

9. The device of claim 1 wherein the arm has a substantially central axis, and the set screw receiving aperture is a bore with a central axis, the arm central axis and the bore central axis being substantially perpendicular to one another.

10. The device of claim 1 wherein the arm has a substantially central axis, and the set screw receiving aperture is a bore with a central axis, the arm central axis and the bore central axis intersecting at an oblique angle.

11. The device of claim 1 wherein the set screw radially extending structure is a projection extending from near a base of the first driving head.

12. The device of claim 11 wherein the projection is a rim.

13. The device of claim 1 wherein the set screw has a flat bottom surface and at least one projection extending from the bottom surface.

14. The device of claim 13 wherein the at least one projection is a central point and a lateral rim.

15. The device of claim 13 wherein the at least one projection is a dome.

16. The device of claim 15 wherein the dome is knurled.

17. A device for connecting a pair of longitudinal connecting members of a spinal implant system, the assembly comprising:
a) a bendable arm;
b) a pair of hook members disposed on either side of the arm, each hook member having a set screw receiving aperture with a first guide and advancement structure thereon and a longitudinal connecting member receiving surface;
b) a pair of set screws, each set screw having a substantially cylindrical body with an axis of rotation and a radially outward surface having a second guide and advancement structure mateable with the first guide and advancement structure, a break-off driving head, a removal head, and a radially extending projection disposed between the break-off head and the removal head; and wherein
c) each receiving surface is oriented with the respective set screw receiving aperture to position a longitudinal connecting member such that a central axis of the longitudinal connecting member substantially intersects with the axis of rotation of the set screw when the longitudinal connecting member is fixed against the receiving surface by the set screw mated with the first guide and advancement structure.

18. The device of claim 17 wherein the hook members are integral with the arm.

19. The device of claim 17 wherein the longitudinal connecting member receiving surfaces are each curvate.

20. The device of claim 17 wherein the break-off driving head has a first polyhedral radially outwardly extending driving surface and the removal head has a second polyhedral radially outwardly extending driving surface.

21. The device of claim 20 wherein the first polyhedral radially outwardly extending driving surface is substantially identical in cross-section to the second polyhedral radially outwardly extending driving surface.

22. The device of claim 20 wherein the radially extending projection is a rim surrounding a base of the first polyhedral radially outwardly extending driving surface.

23. In a transverse connector for detachable engagement with a pair of longitudinal connecting members of a spinal implant system, the connector having an arm, a pair of hooks members, and a pair of set screws receivable in threaded bores of the hook members, the improvement wherein:
a) the hook members are integral with the arm;
b) the arm is bendable;
c) the set screws each have a surface with a projection; and
d) the hook members each have a receiving surface oriented to position a longitudinal connecting member such that a central axis of the longitudinal connecting member substantially intersects with a central axis of the respective threaded bore when a longitudinal connecting member is fixed against the receiving surface by a respective set screw engaged with the threaded bore, with the set screw projection frictionally engaging the longitudinal connecting member.
